(19) United States
(12) Patent Application Publication
(10) Pub. No.: US 2016/0074437 A1
(43) Pub. Date: Mar. 17, 2016

(54) IMMUNOLOGICAL TREATMENT OF LIVER FAILURE

(71) Applicant: Batu Biologics, Inc., San Diego, CA (US)

(72) Inventors: Samuel C. Wagner, San Diego, CA (US); Andy J. Kim, San Diego, CA (US); Hong Ma, San Diego, CA (US); Dimitri Theofilopoulos, San Diego, CA (US)

(21) Appl. No.: 14/852,262

(22) Filed: Sep. 11, 2015

Related U.S. Application Data
(60) Provisional application No. 62/049,013, filed on Sep. 11, 2014.

(51) Int. Cl. A61K 35/28; C12N 5/0775

(52) U.S. Cl. CPC A61K 35/28; C12N 5/0665; C12N 2501/2304; C12N 2501/231; C12N 2501/2313; C12N 2501/232; C12N 2501/15; C12N 2501/165

(57) ABSTRACT

Disclosed are means of treatment of liver failure and augmentation of liver regeneration by utilization of immune modulation through administration of immunocytes and mesenchymal stem cells. In one embodiment liver failure is treated by cord blood mononuclear cells administered allogeneic to the host that have been pretreated with hepatogenic cytokines.

### CROSS-REFERENCE TO RELATED APPLICATION

This application claims the benefit of U.S. Provisional Application No. 62/049,013 filed on Sep. 11, 2014, the contents of which are incorporated by reference herein in its entirety.

### TECHNICAL FIELD

The application pertains to the field of liver failure, more particularly the application pertains to the field of augmenting liver regenerative processes. More particularly, the application pertains to utilization of cells that have immune modulatory properties to stimulate liver regeneration while at the same time reducing liver fibrosis.

### BACKGROUND

Liver failure is a major burden on our health care system and the 7^{th} largest cause of death in industrialized countries. To date the only cure for liver failure is transplantation, which is severely limited by lack of donors and adverse effects of chronic immune suppression. Liver failure is caused as a result of a number of acute and chronic clinical inciting factors, including drug/alcohol-induced hepatotoxicity, viral infections, vascular injury, autoimmune disease, or genetic predisposition [1]. Manifestations of liver failure include fulminant acute hepatitis, chronic hepatitis, or cirrhosis. Subsequent to various acute insults to the liver, the organ regenerates due to its unique self-renewal activity. If the insult is continuously occurring, the liver's capacity to regenerate new cells is overwhelmed and fibrotic non-functional tissue is deposited which takes over the function of the hepatic parenchyma. The subsequent reduction of hepatocyte function can give rise to metabolic instability combined with disruption of essential bodily functions (i.e., energy supply, acid-base balance and coagulation). If not rapidly addressed, complications of hepatic dysfunction such as uncontrolled bleeding and sepsis occur, and dependent organs such as the brain and kidneys cease to function because of accumulation of toxic metabolites. In critical cases, such as when patients progress to Acute-to-Chronic Live Failure (ACLF), liver transplant is considered to be the standard treatment. However, there are often serious difficulties to obtain a suitable donor and many complications arise after transplantation, including rejection and long-term adherence to immunosuppressant regimes.

Although stem cell therapies are currently in development for treatment of liver failure, these possess numerous shortcomings. Embryonic and iPS derived stem cells are all difficult to grow in large quantities and possess the possibility of carcinogenesis or teratoma formation. Additionally, ectopic tissue differentiation in the hepatic microenvironment could have devastating consequences. Adult stem cells offer the possibility of inducing some clinical benefit, however responses to date have not been profound. This is in part because of the inability of adult stem cells to fully take over hepatic tissue. The current invention is based around the notion of using immune modulation, whether by adult stem cells, or by immunocytes, as a means of inhibiting liver failure and inducing regression of disease.

### DETAILED DESCRIPTION OF THE EMBODIMENTS

In one embodiment immunological stimuli are provided to accelerate the process of normal liver regeneration, or to protect the process of normal liver regeneration from fibrosis. It has been demonstrated that up to 70% resection of the liver results in complete regeneration. However this is in situations where there is no inhibition of hepatocyte proliferation. In these situations, the liver depends on proliferation of oval cells.

In another embodiment, immunological intervention is used to allow patients to undergo procedures such as living donor transplantation, two-stage hepatectomies, and split liver transplantation, which would be impossible for patients with various liver pathologies or fibrosis.

There are three phases to liver regeneration that the application teaches an intervention can be made immunologically: a) Priming; b) Proliferation and c) Termination. It is important to note that hepatocytes are not terminally differentiated cells, but cells that reside in a state of proliferative quiescence. Specifically, they share features with other regenerative cells such hematopoietic stem cells, in that they are normally in the GO phase of cell cycle. This is altered during liver regeneration, which is described below.

During the priming phase, numerous injury signals are generated as a result of the underlying injury, and include activators of toll like receptors, complement degradation products, and Damage Associated Molecular Patterns (DAMPs). These signals stimulate various cells, primarily Kupffer cells, to produce cytokines and growth factors such as IL-6, TNF-alpha, and HGF which induce entry of hepatocytes into cell cycle. The importance of these molecular signals in the initiation of liver regeneration is highlighted by knockout studies. Cressmann et al demonstrated in a partial hepatectomy, IL-6 knockout model blockade of liver regeneration that was associated with blunted exit from G0 phase of cell cycle in hepatocytes of these mice, but not in nonparenchymal liver cells. Furthermore, they conclusively showed the importance of IL-6 in that a single preoperative dose of recombinant IL-6 restored post-injury hepatocyte entry into G1/2 to levels observed in wild-type mice and restored biochemical function. NF-kappa B is a major downstream effector of various inflammatory cytokines including TNF-alpha and IL-6. Melato et al generated hepatic specific knockout mice in which the inhibitor of NF-kappa B, IKK2, was ablated, thus giving rise to a higher level of background NF-kappa B activation. In these mice partial hepatectomy resulted in accelerated entry of hepatocytes into cell cycle. The role of a variety of inflammatory or “danger” associated pathways in the initial priming of hepatocyte proliferation after injury has been confirmed using DNA microarray analysis of genes associated with these signaling pathways such as STAT, p38MAPK, and Ras/ERK. The application teaches that depending on patient need, various immunological interventions can be performed at this stage. For example, if the goal of the practitioner is to upregulate extent of hepatocyte regeneration, innate immune stimulators may be administered such as TLR agonists, or BCG, or beta glucan. It is to be noted that these should not be stimulators of robust inflammation that would be deleterious. In one embodiment, stimulators of TLRs may be added together with cells such as mesenchymal stem cells, which would suppress some aspects of the inflammatory response stimulated by TLR activators. In another embodiment, monocytes may be added to the hepatic circulation or intrahepatically in order to augment extent of innate stimulation occurring. In other aspects dendritic cells may be added.

The Proliferation Phase of hepatic regeneration is associated with “primed” hepatocytes leaving G₁ stage of cell cycle and entering S phase, which is accompanied by phosphorylation of the retinoblastoma protein (pRb) and by up-regulated expression of a number of proliferation associated genes including cyclin E, cyclin A, and DNA polymerase . Key cytokines involved in stimulation of proliferation of the hepatocytes include hepatocyte growth factor (HGF) and epidermal growth factor (EGF). HGF is produced by mesenchymal cells, hepatic stellate cells, and liver sinusoidal endothelial cells as a pro-protein, which acts both systemically and locally. Systemic elevations in HGF are observed after partial hepatectomy, whereas local HGF is released from its latent form which is often bound to extracellular matrix proteins. Activation of HGF occurs typically via enzymatic cleavage mediated by urokinase type plasminogen activator (uPA). The importance of HGF in the Proliferation Phase of liver regeneration is observed in animals where the HGF receptor c-MET is conditionally inactivated, which display a reduction in hepatocyte entry into the S phase of cell cycle post injury. EGF signaling has also been demonstrated to be involved in entry into the proliferative phase post injury.

Natarajan et al. performed perinatal deletion of EGFR in hepatocytes prior to partial hepatectomy. They showed that after hepatic injury mice lacking EGFR in the liver had an increased mortality accompanied by increased levels of serum transaminases indicating liver damage. Liver regeneration was delayed in the mutants because of reduced hepatocyte proliferation. Analysis of cell cycle progression in EGFR-deficient livers indicated a defective G(1)-S phase entry with delayed transcriptional activation and reduced protein expression of cyclin D1 followed by reduced cdk2 and cdk1 expression. Immunologically intervening in this stage would require the administration of immune cells producing growth factors. Such cells could be alternatively activated macrophages, or monocytes that have been pretreated with stimuli to increase production of growth factors such as those mentioned above including HGF. One method of stimulating immune cells to produce such growth factors includes culture with IGIV, or stimulation with hypoxia. It is further one embodiment of the invention to stimulate lymphocytes to produce growth factors by various in vitro culture techniques. For example, stimulation of allogeneic or autologous lymphocytes by culture with anti-CD3and anti-CD28 in the presence of hepatocytes can be used to stimulate growth factor production that is beneficial for hepatocyte proliferation in vivo.

The Termination Phase of liver regeneration occurs when the normal liver-mass/body-weight ratio of 2.5% has been restored. While in the Initiation Phase of liver regeneration, several inflammatory cytokines are critical, in the Termination Phase, antiinflammatory cytokines such as IL-10, are upregulated, which dampen proliferative stimuli. Additionally, cytokines with direct antiproliferative activity such as TGF-beta are generated, which result in cell cycle arrest of proliferating hepatocytes. In this phase immunological intervention may be to inhibit the arrest of hepatocyte proliferation, such as utilization of Th17 cells that inhibit TGF-beta production, or administration of cells that are fibrinolytic and express MMPs, such cells include lymphocytes, pretreated lymphocytes, and macrophages. Additionally, administration of MSC that are induced to express MMPs may be performed.

“Treat” or “treatment” means improving the symptoms and ameliorating autoimmune, septic, or pulmonary disease. Additionally, “treat” means improving ischemic conditions. Methods for measuring the rate of “treatment” efficacy are known in the art and include, for example, assessment of inflammatory cytokines.

“Angiogenesis” means any alteration of an existing vascular bed or the formation of new vasculature which benefits tissue perfusion. This includes the formation of new vessels by sprouting of endothelial cells from existing blood vessels or the remodeling of existing vessels to alter size, maturity, direction or flow properties to improve blood perfusion of tissues. As used herein the terms, “angiogenesis,” “revascularization,” “increased collateral circulation,” and “regeneration of blood vessels” are considered as synonymous.

“Chronic wound” means a wound that has not completely closed in twelve weeks since the occurrence of the wound in a patient having a condition, disease or therapy associated with defective healing. Conditions, diseases or therapies associated with defective healing include, for example, diabetes, arterial insufficiency, venous insufficiency, chronic steroid use, cancer chemotherapy, radiotherapy, radiation exposure, and malnutrition. A chronic wound includes defects resulting in inflammatory excess (e.g., excessive production of Interleukin-6 (IL-6), tumor necrosis factor-alpha (TNF-alpha), and MMPs), a deficiency of important growth factors needed for proper healing, bacterial overgrowth and senescence of fibroblasts. A chronic wound has an epithelial layer that fails to cover the entire surface of the wound and is subject to bacterial colonization.

“Cell culture” is an artificial in vitro system containing viable cells, whether quiescent, senescent or (actively) dividing. In a cell culture, cells are grown and maintained at an appropriate temperature, typically a temperature of 37° C. and under an atmosphere typically containing oxygen and CO₂. Culture conditions may vary widely for each cell type though, and variation of conditions for a particular cell type can result in different phenotypes being expressed. The most commonly varied factor in culture systems is the growth medium. Growth media can vary in concentration of nutrients, growth factors, and the presence of other components. The growth factors used to supplement media are often derived from animal blood, such as calf serum.

“Therapeutically effective amount” means the amount of cells, conditioned media or exosomes that, when administered to a mammal for treating a chronic wound, or angiogenic insufficiency is sufficient to effect such treatment. The “therapeutically effective amount” may vary depending on the size of the wound, and the age, weight, physical condition and responsiveness of the mammal to be treated.

“Therapeutic agent” means to have “therapeutic efficacy” in modulating angiogenesis and/or wound healing and an amount of the therapeutic is said to be a “angiogenic modulatory amount”, if administration of that amount of the therapeutic is sufficient to cause a significant modulation (i.e., increase or decrease) in angiogenic activity when administered to a subject (e.g., an animal model or human patient) needing modulation of angiogenesis.

“Growth factor” can be a naturally occurring, endogenous or exogenous protein, or recombinant protein, capable of stimulating cellular proliferation and/or cellular differentiation and cellular migration.

“About” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Unless otherwise stated, the term “about” means within an acceptable error range for the particular value.

“Pharmaceutically acceptable” refers to a natural or synthetic substance means that the substance has an acceptable toxic effect in view of its much greater beneficial effect, while the related the term, “physiologically acceptable,” means the substance has relatively low toxicity.

“Endothelial cell mitogen” means any protein, polypeptide, variant or portion thereof that is capable of, directly or indirectly, inducing endothelial cell growth. Such proteins include, for example, acidic and basic fibroblast growth factors (aFGF) (GenBank Accession No. NP₁₄₉₁₂₇) and bFGF (GenBank Accession No. AAA52448), vascular endothelial growth factor (VEGF) (GenBank Accession No. AAA35789 or NP₀₀₁₀₂₀₅₃₉), epidermal growth factor (EGF) (GenBank Accession No. NP₀₀₁₉₅₄), transforming growth factor-alpha (TGF-alpha) (GenBank Accession No. NP₀₀₃₂₂₇) and transforming growth factor beta (TFG-beta) (GenBank Accession No. 1109243A), platelet-derived endothelial cell growth factor (PD-ECGF) (GenBank Accession No. NP₀₀₁₉₄₄), platelet-derived growth factor (PDGF) (GenBank Accession No. 1109245A), tumor necrosis factor-alpha (TNF-alpha) (GenBank Accession No. CAA26669), hepatocyte growth factor (HGF) (Gen Bank Accession No. BAA14348), insulin like growth factor (IGF) (GenBank Accession No. P08833), erythropoietin (GenBank Accession No. P01588), colony stimulating factor (CSF), macrophage-CSF (M-CSF) (GenBank Accession No. AAB59527), granulocyte/macrophage CSF (GM-CSF) (GenBank Accession No. NP.sub.-000749), monocyte chemotactic protein-1 (GenBank Accession No. P13500) and nitric oxide synthase (NOS) (GenBank Accession No. AAA36365). See, Klagsbrun, et al., Annu. Rev. Physiol., 53:217-239 (1991); Folkman, et al., J. Biol. Chem., 267:10931-10934 (1992) and Symes, et al., Current Opinion in Lipidology, 5:305-312 (1994). Variants or fragments of a mitogen may be used as long as they induce or promote endothelial cell or endothelial progenitor cell growth. Preferably, the endothelial cell mitogen contains a secretory signal sequence that facilitates secretion of the protein. Proteins having native signal sequences, e.g., VEGF, are preferred. Proteins that do not have native signal sequences, e.g., bFGF, can be modified to contain such sequences using routine genetic manipulation techniques. See, Nabel et al., Nature, 362:844 (1993).

“Mesenchymal stem cell” or “MSC” refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. As used herein, “mesenchymal stromal cell” or “MSC” can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. As used herein, “mesenchymal stromal cell” or “MSC” includes cells that are CD34 positive upon initial isolation from tissue but are similar to cells described about phenotypically and functionally. As used herein, “MSC” includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. As used herein, “mesenchymal stromal cell” or “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlioStern®, Astrostem®, lxmyelocel-T, MSC-NTF, NurOwn™ Stemedyne™-MSC, Stempeucel®, StempeucetCLl, StempeuceIOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

While classical liver regeneration is mediated by hepatocytes in certain situations, such as in liver failure, the ability of the hepatocytes to mediate regeneration is limited and liver progenitor cells (LPCs) must carry out the process. The concept of a LPC, which took over regenerative function when hepatocyte multiplication is stunted, was first demonstrated in 1956 when Farber treated rats with various liver carcinogens that blocked division of hepatocytes. He discovered the existence of “Oval Cells” which were subsequently demonstrated to act as LPC having ability to differentiate into both hepatocytes and biliary cells. LPC are found in the canals of Hering and bile ductules in human liver and found increased in patients with chronic liver disease. It is unclear what the origin of LPCs is, whether they derive from local cells, or directly from MSCs, particularly bone marrow derived MSCs, but the cellular mechanisms are poorly understood. In 2000 Theise et al found hepatocytes and cholangiocytes derived from extrahepatic circulating stem cells in the livers of female patients who had undergone therapeutic bone marrow transplantations. In the two female recipients from male donors and four male recipients from female donors, hepatocyte and cholangiocyte engraftment ranged from 4% to 43% and from 4% to 38%, respectively. Given the potent regenerative nature of the liver, combined with the possibility that extrahepatic cellular sources may contribute to regeneration, numerous attempts have been made to utilize cellular therapy for treatment of liver failure. The original hepatic cellular therapies involved the administration of allogeneic hepatocytes, which was attempted in animal models more than 30 years ago and is experimentally used clinically. Unfortunately, major hurdles exist that block this procedures from routine use, specifically: a) low number of suitable donors; b) extremely poor hepatocyte viability after transplantation, with some groups as low as 30%; and c) need for continuous immune suppression which possesses inherent adverse effects. In one embodiment of the invention stimulation of LPC may be performed by administration of immune cells that provide growth factor support for these cells. This includes administration of cord blood mononuclear cells, or monocytes that have been cultured to possess augmented HGF and other hepatogenic growth factors.

It is known in the art that MSC are capable of possessing some activity against liver failure, however these have not been harnessed properly in the clinical setting. One of skill in the art is referred to the examples below of MSC use in liver failure, which the MSC can be manipulated immunologically as described in the current invention to induce optimized therapeutic effects. Mesenchymal stem cells (MSCs) are adult stem cells with self-renewing abilities and have been shown to differentiate into a wide range of tissues including mesoderm- and nonmesoderm-derived, such as hepatocytes. MSCs are capable of entering and maintaining satellite cell niches, particularly in hematopoiesis, and are key in tissue repair and regeneration, aging, and regulating homeostasis. In the case of liver failure, MSCs can aid in regeneration of hepatic tissue, and their interactions with the immune system have potential as adjuvants during organ transplants, including liver transplantation.

MSCs were discovered in 1970 by Friedenstein et al who demonstrated that bone marrow (BM) contained both hematopoietic stem cells (HSCs), which are non-plastic adherent, and a population of a more rare adherent cell. The adherent cells were able to form single cell colonies and were referred to as stromal cells. Those stromal cells, which are capable of self-renewal and expansion in culture are now referred to as mesenchymal stem cells (MSCs). Friedenstein was the first to show that MSCs could differentiate into mesoderm and to demonstrate their importance in controlling the hematopoietic niche.

In the 1980s, more research on MSCs found that they could differentiate into muscle, cartilage, bone and adipose-derived cells. Caplan et al showed that MSCs are responsible for bone and cartilage regeneration induced by local cuing and genetic potential.

In the 1990s, Pittenger et al isolated MSCs from bone marrow and found that they retained their multilineage potential after expanding into selectively differentiated adipocytic, chondrocytic, or osteocytic lineages. Likewise, Kopen et al showed that bone marrow MSCs differentiated into neural cells when exposed to the brain microenvironment. In 1999, Petersen et al found that bone marrow-derived stem cells could be a source of hepatic oval cells in a rat model. Specifically, they used male to female bone marrow transplant and subsequently induced blockade of hepatocyte proliferation by administration of a hepatotoxin followed by partial hepatectomy. As previously described, this procedure stimulates proliferation of LPC or “reserve cells” which generate new hepatocytes, such cells having previously identified as oval cells. Subsequent to the hepatectomy, Y chromosome, dipeptidyl peptidase IV enzyme, and L21-6 antigen were used to identify the newly generated oval cells, and their hepatocytic progeny to be of bone marrow origin.

The first decade of the 21^{st} century saw a surge of research on MSCs, leading to a greater understanding of their nature and of the cellular process behind regeneration. In 2005, Teratani et al identified growth factors allowing hepatic fate-specification in mice and showed that embryonic stem cells could differentiate into functional hepatocytes. In 2007, Chamberlain et al generated human hepatocytes from clonal MSCs in fetal sheep hepatic tissue, differentiating into hepatocytes both throughout the liver parenchyma and the periportal space. The attractive features of MSC for clinical development include their ease of expansion, lack of need for donor matching, and standardized protocols for manufacturing and administration. Of particular interest for liver conditions is the observation that intravenous administration of MSC results in a primary homing of cells to the lung, followed by homing and retention to liver. A unique property of MSC is their apparent hypoimmunogenicity and immune modulatory activity, which is present in MSC derived from various sources. This is believed to account for the ability to achieve therapeutic effects in an allogeneic manner. Allogeneic bone marrow derived MSC have been used by academic investigators with clinical benefit treatment of diseases such as graft versus host (GVHD), osteogenesis imperfecta, Hurler syndrome, metachromatic leukodystrophy, and acceleration of hematopoietic stem cell engraftment. The company Athersys has successfully completed Phase I safety studies using allogeneic bone marrow MSCs is now in efficacy finding clinical trials (Phase II and Phase III) for Multiple Sclerosis, Crohn's Disease, and Graft Versus Host Disease using allogeneic bone marrow derived MSC. Intravenous administration of allogeneic MSCs by Osiris was also reported to induce a statistically significant improvement in cardiac function in a double-blind study.

Currently there several MSC-based therapies that have received governmental approvals including Prochymal™ which was registered in Canada and New Zealand for treatment of graft versus host disease. Although in terms of clinical translation bone marrow MSC are the most advanced, several other sources of MSC are known which possess various properties that may be useful for specific conditions. Bone marrow is also a source for hematopoietic stem cells (HSCs), which have also been used for liver regeneration. Likewise, human placenta is an easily accessible source of abundant MSCs, which can be differentiated in vitro. Finally, MSCs with tissue regenerative abilities can also be isolated from adipose tissue and induced to hepatocytes in large numbers.

Early studies suggested that out of the hepatic regenerative cells found in the bone marrow, that the MSC component is the most regenerative cell type as compared to other cell types such as hematopoietic stem cells. Given the fact that BM-MSC are capable of differentiating into various tissues in vitro, combined with the putative bone marrow origin of the hepatic-repairing oval cell, investigators sought to determine whether BM-MSC could be induced to differentiate into hepatocyte cells in vitro through culture in conditions that would imitate hepatic regeneration. Lee et al developed a 2-step protocol for hepatocyte differentiation using culture in hepatocyte growth factor, followed by oncostatin M. After 4 weeks of induction the investigators reported the spindle-like BM-MSC taking a cuboidal morphology, which is characteristic of hepatocytes. Furthermore the differentiating cells were seen to initiate expression of hepatic-specific genes in a time-dependent manner correlating with morphological changes. From a functional perspective, the generated hepatocytes exhibited features of liver cells, specifically albumin production, glycogen storage, urea secretion, uptake of low-density lipoprotein, and phenobarbital-inducible cytochrome P450 activity. To improve yield and potency of BM-MSC generated hepatocytes, Chen et al utilized conditioned media from cultured hepatocytes as part of the differentiation culture conditions. They reported that BM-MSC cultures in the differentiation conditions started taking an epithelioid, binucleated morphology at days 10 and 20. Gene assessment revealed increase in AFP, HNF-3beta, CK19, CK18, ALB, TAT, and G-6-Pase mRNA, which was confirmed at the protein levels. Additionally, the cells started taking a functional phenotype similar to hepatocytes, including, hepatocyte-like cells by culture in conditioned medium further demonstrated in vitro functions characteristic of liver cells, including glycogen storage, and urea secretion activities. In vivo relevance of these artificially generated hepatic like cells was seen in that restoration of albumin activity and suppression of liver enzymes was seen upon transplantation to immune deficient animals exposed to chemically induced liver injury. In accordance with the concept that injured tissue mediates MSC activation and subsequent repair, Mohsin et al demonstrated that coculture of BM-MSC with chemically-injured hepatocytes augments hepatic differentiation as compared to coculture with naïve hepatocytes.

Based on in vitro differentiation, as well as the possibility of MSC producing cytokines such as HGF, which are known to stimulate hepatic regeneration/decrease hepatocyte apoptosis several animal studies were conducted using BM-MSC in models of liver injury. Fang et al utilized a carbon tetrachloride induced hepatic injury model to assess effects of systemically administered BM-MSC on fibrosis and hepatocyte demise. It was found that MSC infusion after exposure to the hepatotoxin significantly reduced liver damage and collagen deposition. Supporting the possibility of a concurrent protective and regenerative effect, levels of hepatic hydroxyproline and serum fibrosis markers in mice receiving cells were significantly lower compared with those of control mice. Histologic examination suggested that hepatic damage recovery was accelerated in the treated mice. Donor cell engraftment and possible in vivo hepatic differentiation was supported by immunofluorescence, polymerase chain reaction, and fluorescence in situ hybridization analysis, which demonstrated donor-derived cells possessing epithelium-like morphology expressed albumin. Interestingly, the amount of engrafted cells was minute and could not explain the functional recovery of serum albumin, suggesting the possibility of paracrine effects. A subsequent study using the same carbon tetrachloride model demonstrated that BM-MSC administration resulted to reactive oxygen species ex vivo, reduced oxidative stress in recipient mice, and accelerated repopulation of hepatocytes after liver damage. To optimize the route of administration, Zhao et al assessed intravenous, intrahepatic, and intraperitoneal administration of BM-MSC in rats treated with carbon tetrachloride. Functional recovery was most profound in the intravenous administration group, which was correlated with increased IL-10 and decreased IL-1, TNF-alpha, and TGF-beta. Furthermore, in vivo differentiation of the BM-MSC was observed based on expression of α-fetoprotein, albumin, and cytokeratin 18 in cells deriving from donor origin.

In order to assess whether the therapeutic effects of BM-MSC are specific to the carbon tetrachloride model, or whether they may be extrapolated to other models of hepatic injury, investigators assessed whether BM-MSC are useful in hepatectomy recovery models. While recovery is generally observed after ⅔ or 70% hepatectomy, 90% hepatectomy is lethal in rats. In one study, BM-MSC were differentiated in vitro by culture on Matrigel with hepatocyte growth factor and fibroblast growth factor-4 into cells expressing hepatocyte-like properties. Specifically, the cells expressed a hepatic-like cuboidal morphology and were positive for albumin, cytochrome P450 (CYP) 1A1, CYP1A2, glucose 6-phosphatase, tryptophane-2,3-dioxygenase, tyrosine aminotransferase, hepatocyte nuclear factor (HNF)1 alpha, and HNF4alpha. Intrasplenic administration of differentiated cells subsequent to the 90% hepatectomy resulted prevention of lethality. Another study confirmed efficacy of BM-MSC at accelerating post-hepatectomy liver regeneration subsequent to intraportal administration. Regenerative effects where associated with upregulation of HGF expression in the newly synthesized tissue. It is interesting that the regenerative effects of BM-MSC are observed not only in acute settings but also in chronic conditions leading to liver failure. Non-alcoholic steatohepatitis (NASH) is a precursor to cirrhosis and is characterized by lipid accumulation, hepatocyte damage, leukocyte infiltration, and fibrosis. It was demonstrated that in C57BL/6 mice chronically fed with high-fat diet, that the intravenous administration of BM-MSC resulted in reduction of plasma levels of hepatic enzyme, hepatomegaly, liver fibrosis, inflammatory cell infiltration, and inflammatory cytokine gene expression, as compared to control mice. Overall, these data suggest that BM-MSC has some repairative/regenerative activity on livers that are damaged in either chronic or acute settings.

Additional animal studies were conducted in both chronic and acute liver toxicity settings. For example, Hwang et al, treated Sprague-Dawley rats with 0.04% thioacetamide (TAA)-containing water for 8 weeks, and BM-MSC were injected into the spleen with the intent of transsplenic migration into the liver. Ingestion of TAA for 8 weeks induced micronodular liver cirrhosis in 93% of rats. Examination of MSC microscopically revealed that the injected cells were diffusely engrafted in the liver parenchyma, differentiated into CK19 (cytokeratin 19)—and thy1-positive oval cells and later into albumin-producing hepatocyte-like cells. MSC engraftment rate per slice was measured as 1.0-1.6%. MSC injection resulted in apoptosis of hepatic stellate cells and resultant resolution of fibrosis, but did not cause apoptosis of hepatocytes. Given that stellate cells are responsible for matrix deposition and fibrosis, this is an interesting observation. Injection of MSCs treated with HGF in vitro for 2 weeks, which became CD90-negative and CK18-positive, resulted in chronological advancement of hepatogenic cellular differentiation by 2 weeks and decrease in anti-fibrotic activity. Mechanistically, it appeared that the BM-MSC directly differentiated to oval cells and hepatocytes, which was associated with repair of damaged hepatocytes, intracellular glycogen restoration and resolution of fibrosis.

An acute model of liver failure is produced by administration to animals of D-galactosamine, a TNF-alpha stimulating hepatotoxin, and lipopolysaccharide (LPS) a potent inflammatory stimulus that replicates translocation of gut bacteria often seen in liver failure. In this model it was demonstrated that administration of BM-MSC in pretreated rats resulted in reduction of ALT, AST, caspase-1 and IL-18 proteins, and mRNA as compared to the control group. Mechanistic elucidation at a cellular level demonstrated that the injected BM-MSC were inhibiting hepatocyte apoptosis. Interestingly the authors also found that recovering animals possessed higher levels of VEGF protein as compared to non-treated animals. This is intuitively logical given that VEGF is a key cytokine in the angiogenesis cascade, and angiogenesis seems to be required regression of liver failure. Using the same D-galactosamin/LPS model, Sun et al, sought to identify optimal route of delivery for BM-MSC. They divided rats into the following groups: a) hepatic artery injection; b) portal vein injection; c) tail vein injection group; and d) intraperitoneal injection. They found that compared with the control group, ALT, AST, and damage to the liver tissue in the hepatic artery group, the portal vein group and the tail vein group improved in vivo. The expression of PCNA and HGF in the liver was higher and caspase-3 expression was lower in the hepatic artery injection group, the portal vein injection group and the tail vein injection group than that in the intraperitoneal injection and control groups. The BRdU-labeled BM-MSCs were only observed homing to the liver tissue in these three groups. However, no significant differences were observed between these three groups. Liver function was improved following BM-MSC transplantation via 3 endovascular implantation methods (through the hepatic artery, portal vein and vena caudalis).

These data suggest that intra-hepatic artery injection was most effective and that intraperitoneal administration is ineffective. A large animal study using similar hepatotoxins was performed in the pig. Li et al. administered 3×10(7) human BM-MSC via the intraportal route or peripheral vein immediately after D-galactosamine injection, and a sham group underwent intraportal transplantation (IPT) without cells (IPT, peripheral vein transplantation [PVT], and control groups, respectively, n=15 per group). All of the animals in the PVT and control groups died of FHF within 96 hours. In contrast, 13 of 15 animals in the IPT group achieved long-term survival (>6 months). Immunohistochemistry demonstrated that transplanted human BM-MSC-derived hepatocytes in surviving animals were widely distributed in the hepatic lobules and the liver parenchyma from weeks 2 to 10. Thirty percent of the hepatocytes were BM-MSC-derived. However, the number of transplanted cells decreased significantly at week 15. Only a few single cells were scattered in the regenerated liver lobules at week 20, and the liver tissues exhibited a nearly normal structure. These data suggest that intraportal delivery may be ideal and also reinforce the notion that MSC may be transplanted across allo and xeno barriers without need for immune suppression.

Clinical trials utilizing BM-MSC have shown an excellent safety profile, with various levels of efficacy in liver failure. Mohamadnejad et al , conducted a 4 patient study with decompensated liver cirrhosis. Patoemt bone marrow was aspirated, mesenchymal stem cells were cultured, and a mean 31.73×10(6) mesenchymal stem cells were infused through a peripheral vein. There were no side-effects in the patients during follow-up. The model for end-stage liver disease (MELD) scores of patients 1, and 4 improved by four and three points, respectively by the end of follow-up. Furthermore, the quality of life of all four patients improved by the end of follow-up. Using SF-36 questionnaire, the mean physical component scale increased from 31.44 to 65.19, and the mean mental component scale increased from 36.32 to 65.55. Another study treated 8 patients (four hepatitis B, one hepatitis C, one alcoholic, and two cryptogenic) with end-stage liver disease having MELD score > or =10 were included. Autologous BM-MSCs were taken from iliac crest. Approximately, 30-50 million BM-MSCs were proliferated and injected into peripheral or the portal vein. Subsequent to experiment the MELD Score was decreased from 17.9 +/−5.6 to 10.7 +/−6.3 (P<0.05) and prothrombin complex from international normalized ratio 1.9 +/−0.4 to 1.4 +/−0.5 (P<0.05). Serum creatinine decreased from 114 +/−35 to 80 +/−18 micromol/l (P<0.05). This trial supports the safety with signal of efficacy of the BM-MSC activity in liver failure clinically.

A larger trial of autologous BM-MSC focused on patients with liver failure associated with hepatitis B infection. Part of the rational was previous studies showing that BM-MSC derived hepatocytes are resistant to hepatitis B infection. Peng et al, treated 53 patients and as controls used 105 patients matched for age, sex, and biochemical indexes, including alanine aminotransferase (ALT), albumin, total bilirubin (TBIL), prothrombin time (PT), and MELD score. In the 2-3 week period after cell administration, efficacy was observed based on levels of ALB, TBIL, and PT and MELD score, compared with those in the control group. Safety of the procedure was demonstrated in that there were no differences in incidence of hepatocellular carcinoma (HCC) or mortality between the treated and control groups at 192 weeks. Unfortunately, liver function between the two groups was also similar at 192 weeks, suggesting the beneficial effects of BM-MSC were transient in nature. Supporting the possibility of transient effects of BM-MSC was a 27 patient study in patients with decompensated cirrhosis in which 15 patients received BM-MSC and 12 patients received placebo. The absolute changes in Child scores, MELD scores, serum albumin, INR, serum transaminases and liver volumes did not differ significantly between the MSC and placebo groups at 12 months of follow-up. Unfortunately the publication did not provide 3 or 6 month values.

In contrast, a more recent study administered BM-MSC into 12 patients (11 males, 1 female) with baseline biopsy-proven alcoholic cirrhosis who had been alcohol free for at least 6 months. A 3 month assessment histological improvement and reduction of fibrosis was quantified according to the Laennec fibrosis scoring scale in 6 of 11 patients. Additionally, at 3 months post cell administration, the Child-Pugh score improved significantly in ten patients and the levels of transforming growth factor-β1, type 1 collagen and a-smooth muscle actin significantly decreased (as assessed by real-time reverse transcriptase polymerase chain reaction) after BM-MSCs therapy. Overall the different underlying conditions, route of administration, and time points of assessments between studies makes it difficult to draw solid conclusions, although it appears that some therapeutic effect exists, although longevity of effect is not known.

Given that one possibility for the lack of efficacy long term in the previous study may be inappropriate level of hepatocyte differentiation in vivo, Amer et al conducted a clinical trial where BM-MSC were pre-differentiated toward the hepatocyte lineage by a culture cocktail containing HGF They conducted a 40 patient trial in hepatitis C patients in which 20 patients were treated with partially differentiated cells either intrasplenically or intrahepatically and 20 patients received placebo control. At the 3 and 6 month time points a significant improvement in ascites, lower limb edema, and serum albumin, over the control group was observed. Additionally significant benefit was quantified in the Child-Pugh and MELD scores. No difference was observed between intrahepatic or intrasplenic administration. This study demonstrates the potential of semi-differentiated hepatocytes from BM-MSC to yield therapeutic benefit without reported adverse effects.

Out of the BM-MSC studies described, one potential reason for relatively mediocre results could be the fact that autologous cells where utilized in all of the studies. While autologous MSC possess the benefit of lack of immunogenicity, a drawback may be a relative dysfunction of these cells given the poor health condition of the patients. Indeed several studies have demonstrated that MSC from patients suffering from chronic conditions possess inhibited regenerative activity when compared with MSC from healthy donors.

Adipose tissue is an attractive alternative to bone marrow as a source of stem cells for treatment of degenerative conditions in general and liver failure specifically, for the following reasons: a) extraction of adipose derived cells is a simpler procedure that is much less invasive than bone marrow extraction; b) Adipose tissue contains a higher content of mesenchymal stem cells (MSC) as compared to bone marrow, therefore shorter in vitro expansion times are needed; and c) MSC from adipose tissue do not decrease in number with aging. Adipose tissue derived MSC were originally described by Zuk et al who demonstrated the stromal vascular fraction (SVF) of adipose tissue contains large numbers of cells that could be induced to differentiate into adipogenic, chondrogenic, myogenic, and osteogenic lineages and morphologically resembled MSC. Subsequent to the initial description, the same group reported that in vitro expanded SVF derived cells had surface marker expression similar to bone marrow derived MSC, displaying expression of CD29, CD44, CD71, CD90, CD105/SH2, and SH3 and lacking CD31, CD34, and CD45 expression. This suggested that SVF expanded adherent cells where indeed members of the MSC family, a notion that has subsequently gained acceptance. To date, clinical trials on adipose derived cells have all utilized ex vivo-expanded cells, which share properties with bone marrow derived MSC. Preparations of MSC expanded from adipose tissue are equivalent or superior to bone marrow in terms of differentiation ability, angiogenesis-stimulating potential [153], and immune modulatory effects. This invention provides the use of endothelial cell mitogens together with various MSC sources such as AT-MSC, preferably in the autologous setting and Wharton's Jelly MSC in the allogeneic setting. Additionally, as MSC may be pretreated with a stressor before administration for therapeutic effects.

In the area of liver failure Banas et al created a 13 day in vitro differentiation protocol to generate hepatocyte like cells from human adipose tissue MSC (AT-MSC). The differentiated cells possessed a hepatocyte-like morphology and phenotypically resembled primary hepatocytes. Administration of the cells in a carbon tetrachloride induced liver failure model resulted in diminished liver injury, AST, ALT, as well as ammonia. Unfortunately comparison with BM-MSC was not performed. A subsequent study utilized AT-MSC that were not differentiated and injected into the tail vein. Administration of cells led to death in 4 of 6 mice due to lung infarction, presumably as a result of cell accumulation in pulmonary microcapillaries. To overcome this the investigators utilized a combination of AT-MSC and heparin, this resulted in trend, which did not reach significance, for reduced ALT, AST, and LDH in the treated group. It was demonstrated in a subsequent tracking study by the same group that heparin decreased pulmonary retention and increased hepatic retention by 30%.

In order to elucidate whether alternative routes of AT-MSC administration may augment therapeutic activities, Kim et al, assessed intravenous, intrahepatic parenchyma, and intra-portal vein delivery of cells in the same carbon tetrachloride model as utilized by the previous two experiments. They found that all 3 routes led to significant decrease in histological injury as well as AST, ALT, and ammonia. The most profound protective effects where observed with the intravenous route was used. One possible reason for statistical significant efficacy in this study and not in the previous study may be that in this study AT-MSC were injected at day 1 and 3 after carbon tetrachloride administration, whereas the previous study involved only one injection. While the previous AT-MSC experiments utilized human cells administered in animals, Deng et al, utilized syngeneic AT-MSC that were derived from mice transgenic with enhanced green fluorescent protein (eGFP) in mice treated with carbon tetrachloride. The survival rate of cell treated group significantly increased compared to PBS group. Furthermore, the transplanted cells were well integrated into injured livers and produced albumin, cytokeratin-18. Overall, it appears that in the carbon tetrachloride model both xenogeneic and syngenic AT-MSC have therapeutic effects, however standardization of protocols and models is needed to obtain a clearer picture of potency of effects.

In one embodiment of the invention, MSC are utilized together with endothelial cells, or endothelial progenitor cells to accelerate the process of liver regeneration or to induce regression of fibrotic tissues.

Other models of hepatic injury have been utilized with AT-MSC. Salomone et al assessed human AT-MSC transfected with eGFP in rats treated with a hepatoxic dose of acetaminophen. It was found that AT-MSC infusion decreased AST, ALT and prothrombin time to the levels observed in control rats. Furthermore clinical signs of liver failure such as encephalopathy were not observed in treated animals. Histologically, control animals displayed lobular necrosis and diffuse vacuolar degeneration, which was not seen, in the treated group. Mechanistically, transplanted AT-MSC induced an increase in antioxidant status and decrease in inflammatory cytokines in the recipients. Additionally, proliferation of endogenous hepatocytes was observed. Indeed it is within the context of the current invention to transfect AT-MSC with immune modulatory genes in the same or similar way that the authors of reference transfected AT-MSC with eGFP gene and to use them for immune modulation. Selected genes that are useful for the practice of the invention are dependent on the phase of liver regeneration where modulation is sought. For example if increased priming is sought MSC may be transfected with IL-6, complement components, or TLR activators. If augmentation of the proliferative phase is sought, MSC may be transfected with growth factors such as HGF, VEGF, or PDGF. If stimulation of antifibrotic mechanisms is required, cells may be transfected with various MMPs.

Indeed another study utilized two chemicals that block hepatocyte regeneration together with partial hepatectomy. Specifically, using a model of a toxic liver damage in Sprague Dawley rats, generated by repetitive intraperitoneal application of retrorsine and allyl alcohol followed by two third partial hepatectomy, investigators assessed the regenerative effects of human AT-MSC. Six and twelve weeks after hepatectomy, animals were sacrificed and histological sections were analyzed. AT-MSC treated animals exhibited significantly raised albumin, total protein, glutamic oxaloacetic transaminase and LDH. The infused cells were found up to twelve weeks after surgery in histological sections. Although to our knowledge clinical studies of AT-MSC in liver disease have not been reported, one clinical trial (NCT01062750) is reported to be enrolling. This trial, run by Shuichi Kaneko of Kanazawa University in Japan comprises of intra-hepatic administration of AT-MSC.

Although numerous studies have examined the ability of MSC to induce hepatic regeneration, the original studies that demonstrated BM liver regenerative effects suggested that other cells in the BM compartment besides MSC may have therapeutic activities. Given that bone marrow mononuclear cells (BMMC) have demonstrated therapeutic activities in numerous ischemic and chronic conditions, investigators sought to assess whether this mixture of cells would possess activity in animal models of liver failure. Terai et al administered BMMC isolated from mice transgenic for GFP to mice whose livers where injured by carbon tetrachloride. It was observed that the transplanted GFP-positive BMMC migrated into the pen-portal area of liver lobules after one day, and repopulated as much as 25% of the recipient liver by 4 weeks. Interestingly when mice where administered BMMC but not carbon tetrachloride, no donor cells could be detected at 4 weeks, indicating that injury must be present for long term hepatic retention. It appeared that the transplanted BMMC differentiated into functional mature hepatocytes which would overtake function of hepatocytes from carbon tetrachloride injured mice. A subsequent study by the same group examined mechanisms of the antifibrotic/regenerative effect of the BMMC and found matrix metalloprotease (MMP) activation to be involved. MMPs are important in liver regeneration not only because of their ability to cleave through fibrotic tissue in order to alter the local environment, but also because of their role in angiogenesis, which is important for liver regeneration. Accordingly, the combination of agents that stimulate MMP expression together with MSC within the context of this invention as a therapeutic mixture.

One of the first clinical uses of BMMC in the liver involved purification of CD133 positive cells prior to administration, with the notion that CD133 selects for cells with enhanced regenerative potential. Additionally, the CD133 subset of bone marrow cells may represent a hepatogenic precursor cell since cells of this phenotype are mobilized from the bone marrow subsequent to partial hepatectomy. Another interesting point is that CD133 has been reported by some to be expressed on oval cells in the liver, although the bone marrow origin is controversial. In 2005 Esch et al described 3 patients subjected to intraportal administration of autologous CD133(+) BMSCs subsequent to portal venous embolization of right liver segments, used to expand left lateral hepatic segments. Computerized tomography scan volumetry revealed 2.5-fold increased mean proliferation rates of left lateral segments compared with a group of three consecutive patients treated without application of BMSCs. In 2012 the same group reported on 11 patients treated with this procedure and 11 controls. They reported that mean hepatic growth of segments II/III 14 days after portal vein embolization in the group that received CD133 cells was significantly higher (138.66 mL±66.29) when compared with the control group (62.95 mL±40.03; P=0.004). Post hoc analysis revealed a better survival for the group that received cells as compared to the control.

A similar study by another group involved 6 patients receiving CD133 cells to accelerate left lateral segment regeneration, with 7 matched control patients. The increase of the mean absolute future liver remnant volume (FLRV) in the treated group from 239.3 mL +/−103.5 to 417.1 mL +/−150.4 was significantly higher than that in the control group, which was from 286.3 mL +/−77.1 to 395.9 mL +/−94.1. The daily hepatic growth rate in the treated group (9.5 mL/d +/−4.3) was significantly higher to that in the control group (4.1 mL/d +/−1.9) (P=0.03). Furthermore, time to surgery was 27 days +/−11 in the treated group and 45 days +/−21 in the control group (P=0.057). These data suggest that in the clinical situation, CD133 cells isolated from BMMC appear to accelerate liver regeneration.

Another purified cell type from BMMC is CD34 expressing cells, which conventionally are known to possess the hematopoietic stem cell compartment. Additionally, similar to CD133, CD34 is found on oval cells in the liver, suggesting possibility that bone marrow derived CD34 cells play a role in liver regeneration when hepatocyte proliferation is inhibited. Gordon et al, reported 5 patients with liver failure that were treated with isolated CD34 positive cells. Interestingly, instead of collected the cells from bone marrow harvest, the investigators mobilized the bone marrow cells by treatment with G-CSF. The investigators first demonstrated that these CD34 cells were capable of differentiating in vitro into albumin producing hepatocyte-like cells. A pilot clinical investigation was attempted in 5 patients with liver failure. The CD34 cells were injected into the portal vein (three patients) or hepatic artery (two patients). No complications or specific side effects related to the procedure were observed. Three of the five patients showed improvement in serum bilirubin and four of five in serum albumin. A subsequent publication by the same group reported the improvement in bilirubin levels was maintained for 18 months. A subsequent case report by Gasbarrini et al [185]. described use of autologous CD34⁺ BMMC administered via the portal vein as a rescue treatment in an alcoholic patient with nimesulide-induced acute liver failure. A liver biopsy performed at 20 days following infusion showed augmentation of hepatocyte replication around necrotic foci; there was also improvement in synthetic liver function within the first 30 days.

Subsequent to the initial studies on CD133 and CD34 cells, investigators assessed the effects of unpurified BMMC on liver failure. Terai et al, treated 9 patients with liver cirrhosis from a variety of causes with autologous BMMC administered intravenously. Significant improvements in serum albumin levels and total protein were observed at 24 weeks after BMMC therapy. Significantly improved Child-Pugh scores were seen at 4 and 24 weeks. alpha-Fetoprotein and proliferating cell nuclear antigen (PCNA) expression in liver biopsy tissue was significantly elevated after BMMC infusion. No major adverse effects were noted. A subsequent study in alcohol associated decompensated liver failure examined effects of autologous BMMC administered intraportally in 28 patients compared to 30 patients receiving standard medical care. After 3 months, 2 and 4 patients died in the BMMC and control groups, respectively. Adverse events were equally distributed between groups. The MELD score improved in parallel in both groups during follow-up. Comparing liver biopsy at 4 weeks to baseline, steatosis improved, and proliferating HPC tended to decrease in both groups. It is unclear why this larger study generated a negative outcome compared to the initial smaller study.

Interestingly in another study in which 32 patients with decompensating liver cirrhosis were treated with autologous BMMC and 15 patients received standard of care, significant improvements were observed. Specifically, improvements in ALT, AST, albumin, bilirubin and histological score where observed. The efficacy of BMMC transplantation lasted 3-12 months as compared with the control group. Serious complications such as hepatic encephalopathy and spontaneous bacterial peritonitis were also significantly reduced in BM-MNCs transfused patients compared with the controls. However, these improvements disappeared in 24 months after transplantation [187]. It is possible that effects of BMMC are transient in liver failure, lasting less than 12 months. For example, Lyra et al, reported on 10 patients with Child-Pugh B and C liver failure who received autologous BMMC. Bilirubin levels were lower at 1 (2.19 +/−0.9) and 4 months (2.10 +/−1.0) after cell transplantation that baseline levels (2.78 +/−1.2). Albumin levels 4 months after BMMC infusion (3.73 +/−0.5) were higher than baseline levels (3.47 +/−0.5). International normalized ratio (INR) decreased from 1.48 (SD=0.23) to 1.43 (SD=0.23) one month after cell transplantation. A larger study by the same group utilizing similar methodology reported similar transient benefit. Specifically, a 30 patient study was conducted with hepatic cirrhosis patients on the transplant list who were randomized to receive BMMC or supportive care. Child-Pugh score improved in the first 90 days in the cell therapy group compared with controls. The MELD score remained stable in the treated group but increased during follow-up in the control group. Albumin levels improved in the treatment arm, whereas they remained stable among controls in the first 90 days. Bilirubin levels increased among controls, whereas they decreased in the therapy arm during the first 60 days; INR RC differences between groups reached up to 10%. The changes observed did not persist beyond 90 days.

Other means of utilizing bone marrow stem cells for hepatic regeneration include stimulating mobilization of endogenous stem cells by providing agents such as G-CSF. Experimental studies to investigate the mobilization of HSCs for hepatocyte formation have yielded conflicting results, but Shitzu et al in 2012 showed beneficial effects in a murine model of acute liver failure.

Several experimental studies have shown that MSCs isolated from human placenta promote healing in diseased rat livers, with an anti-fibrotic effect in liver cirrhosis or reduction of fibrotic tissue. By transplanting placenta-derived MSCs in the portal vein, Cao et al observed promising results in pigs, not only by producing hepatocytes but also by prolonging survival time, reducing necrosis and promoting regeneration.

Another fetal associated tissue that has demonstrated to be a potent source of MSC is umbilical cord. Shi et al, utilized umbilical cord-derived MSC (UC-MSC) administration to treat acute on chronic liver failure (ACLF) patients that had HBV infection. Twenty four patients were treated with UC-MSCs, and 19 patients were treated with saline as controls. The UC-MSC transfusions significantly increased the survival rates in the patients; diminished MELD score; increased serum albumin, cholinesterase, prothrombin activity; and increased platelet counts. Serum total bilirubin and ALT levels were significantly decreased after the UC-MSC at 48 and 72 weeks.

Various populations of mesenchymal stem cells may be used for the practice of the invention, in addition to bone marrow, adipose, or umbilical cord derived mesenchymal stem cells, amniotic membrane mesenchymal stem cells may be utilized as immune modulatory cells. In one specific embodiment, 8 8 cm² sections of amniotic membrane are obtained. They were washed with 1.0M phosphate-buffered saline (PBS; pH 7.2) containing 300 IU/ml penicillin and 300 mg/ml streptomycin (Gibco, Grand Island, N.Y., USA), and are immediately immersed in Dulbecco's modified Eagle's medium (DMEM)-high glucose (Gibco), supplemented with 10% fetal bovine serum (FBS; Gibco), 300 IIU/ml penicillin and 300 mg/ml streptomycin. All samples are processed within 12-15 h after collection. The amniotic membranes are treated with 0.1% collagenase I (Sigma-Aldrich, St Louis, Mo., USA) in 1.0M PBS (pH 7.2) and are incubated at 37° C. for 20 min. Each amniotic membrane is washed three times with low-glucose DMEM (Gibco), and the detached cells are harvested after a gentle massage of the amniotic membrane. The cells are centrifuged at 300 g for 10 min at 37° C., and subsequently resuspended in RPMI 1640 medium with 10% FBS, then grown in 25 cm² flasks at a density of 1 to 106 cells/ml. After 24 h incubation, nonadherent cells are removed. The culture medium is replaced every 3 days. Adherent cells are cultured until they reached 80-90% confluence. Cells are subsequently selected based on quality control procedures including purity (eg >90% CD90 and CD105 positive), sterility (eg lack of endotoxin and mycoplasma/bacterial contamination) and potency (eg ability to immune modulate in vitro by suppressing production of inflammatory cytokines such as IFN-gamma).

Cells may subsequently be utilized for perilymphatic or intralymphatic administration. The present application contemplates the collection and delivery of a naturally occurring population of MSC derived from intra alia, placental/umbilical cord, bone marrow, skin, or tooth pulp tissue. In accordance with the invention, the MSCs are generally an adherent cell population expressing markers CD90 and CD105 (>90%) and lacking expression of CD34 and CD45 and MHC class II (<5%) as detected by flow cytometry, although other markers described in the specification may be utilized.

In the case of placental tissue, which represents an almost unlimited supply of MSC, placenta are collected from delivery procedures, the tissue may be placed in sterile containers with phosphate buffered saline (“PBS”), penicillin/streptomycin and amphotericin B during collection. This may be performed when collecting testicular or ovarian tissue as well. Specifically, harvested tissue is first surface sterilized by multiple washes with sterile PBS, followed by immersion in 1% povidoneiodine (“PVP-1”) for approximately 2 minutes, immersion in 0.1% sodium thiosulfate in PBS for approximately 1 minute, and another wash in sterile PBS.

Next the tissue is dissected into 5 g pieces for digestion. Enzymatic digestion is performed using a mixture of collagenase type I and type II along with thermolysin as a neutral protease. The digestion occurs in a 50 cc sterile chamber for 20-45 minutes until the tissue is disaggregated and the suspending solution is turbid with cells. Next the solution is extracted leaving behind the matrix, and cold (4° C.) balanced salt solution with fetal bovine serum at 5% concentration is added to quench the enzymes. This resulting suspension is centrifuged at 600.times.g, supernatant is aspirated and MESENCULT.RTM. complete medium (basal medium containing MSC stimulatory supplements available from StemCell Technologies, Vancouver, British Columbia) is added to a final volume of approximately 1.5 times the digestion volume to neutralize the digestion enzymes. This mixture is centrifuged at 500 g for 5 minutes, and the supernatant aspirated. The cell pellet is be re-suspended in fresh 10 MESENCULT.RTM. complete medium plus 0.25 mg/mL amphotericin B, 100 IU/mL penicillin-G, and 100 mg/mL streptomycin (JR Scientific, Woodland, Calif.).

Cells are then plated at an initial concentration of approximately one starting 5 g tissue digest per 225 cm² flask. Culture flasks are monitored daily and any contaminated flasks removed immediately and recorded. Non-contaminated flasks are monitored for cell growth, with medium changes taking place three times per week. After 14 days of growth, MSC are detached using 0.25% trypsin/ImM EDTA (available from Invitrogen, Carlsbad, Calif.). Cell counts and viability were assessed using flow cytometry techniques and cells are banked by controlled rate freezing in sealed vials.

For the preparation of bone marrow MSC, bone marrow is collected and placed within a “washing tube”. Before the collection procedure a “washing tube” is prepared in the class 100 Biological Safety Cabinet in a Class 10,000 GMP Clean Room. To prepare the washing tube, 0.2 mL amphotericin B (Sigma-Aldrich, St. Louis, Mo.), 0.2 mL penicillin/streptomycin (Sigma 50 ug/nl) and 0.1 mL EDTANa2 (Sigma) is added to a 50 mL conical tube (Nunc) containing 40 mL of GMP-grade phosphate buffered saline (PBS). Specifically, the washing tube containing the collected bone marrow is topped up to 50 mL with PBS in a class 100 Biological Safety Cabinet and cells are washed by centrifugation at 500 g for 10 minutes at room temperature, which produced a cell pellet at the bottom of the conical tube. Under sterile conditions supernatant is decanted and the cell pellet is gently dissociated by tapping until the pellet appeared liquid. The pellet is re-suspended in 25 mL of PBS and gently mixed so as to produce a uniform mixture of cells in 30 PBS.

In order to purify mononuclear cells, 15 mL of Ficoll-Paque (Fisher Scientific, Portsmouth N.H.) density gradient was added underneath the cell-PBS mixture using a 15 mL pipette. The mixture is subsequently centrifuged for 20 minutes at 900 g. Thereafter, the buffy coat is collected and placed into another 50 mL conical tube together with 40 mL of PBS. Cells are then centrifuged at 400 g for 10 minutes, after which the supernatant is decanted and the cell pellet re-suspended in 40 mL of PBS and centrifuged again for 10 minutes at 400 g. The cell pellet is subsequently re-suspended in 5 mL complete DMEM-low glucose media (GibcoBRL, Grand Island, N.Y.) supplemented with approximately 20% Fetal Bovine Serum specified to have Endotoxin level less than or equal to 100 EU/mL (with levels routinely less than or equal to 10 EU/mL) and hemoglobin level less than or equal to 30 mg/dl (levels routinely less than or equal to 25 mg/dl). The serum lot used is sequestered and one lot is used for all experiments. Additionally, the media is supplemented with 1% penicillin/streptomycin, 1% amphotericin B, and 1% glutamine. The re-suspended cells are mononuclear cells substantially free of erythrocytes and polymorphonuclear leukocytes as assessed by visual morphology microscopically. Viability of the cells was assessed with trypan blue. Only samples with >90% viability were selected for cryopreservation in sealed vials.

For preparation of MSC from teeth, said teeth are extracted under sterile conditions and placed into sterile chilled vials containing 20 mL of phosphate buffered saline with penicillin/streptomycin and amphotericin B (Sigma-Aldrich, St. Louis, Mo.). Teeth were thereafter externally sterilized and processed first 20 by washing several times in sterile PBS, followed by immersion in 1% povidoneiodine (PVP-1) for 2 minutes, immersion in 0.1% sodium thiosulfate in PBS for 1 minute, followed by another wash in sterile PBS. The roots of cleaned teeth is separated from the crown using pliers and forceps to reveal the dental pulp, and the pulp is placed into an enzymatic bath consisting of type I and type II collagenase (Vitacyte, Indianapolis, USA) with thermolysin as the neutral protease. Pulp tissue is allowed to incubate at 37.degree. C. for 20-40 min to digest the tissue and liberate the cells.

Once digestion is complete, MESENCULT.RTM. complete medium is added to a final volume of 1.5.times. the digestion volume to neutralize the digestion enzymes. This mixture is centrifuged at 500 g for 5 min, and the supernatant aspirated. The cell pellet sare resuspended in fresh MESENCULT.RTM. complete medium plus 0.25 mg/mL amphotericin B, 100 30 IU/mL penicillin-G, and 100 mg/mL streptomycin (JR Scientific, Woodland, Calif.). Cells are plated at an initial concentration of one tooth digest per 25 cm.sup.2 flask. Culture flasks are monitored daily and any contaminated flasks removed immediately and recorded. Non-contaminated flasks were monitored for cell growth, with medium changes taking place three times per week. After 14 days of growth, MSC are detached using 0.25% trypsin/ImM EDTA (Invitrogen, Carlsbad, Calif.), cell counts and viability were assessed using a standard trypan blue dye exclusion assay (Sigma) and hemacytometer, and bAU3 the DPSC divided equally between two 75 cm.sup.2 flasks. After the first passage, DPSC cultures were harvested once they reach 7080% confluence. These cells are then cryopreserved in sealed vials.

MSCs from the skin, including epidermal, dermal, and subcutaneous tissue of healthy adult patients undergoing cosmetic plastic surgery are isolated by collagenase digestion procedure. Once received, the tissue is cleaned of any unwanted adipose tissue and hair The tissue is then sterilized using 1× PVP-iodine solution and 1× sodium thiosulfate followed by washing twice in sterile PBS. The dermis is then minced into 1 mm³ pieces following collagenase enzymatic digestion for 30-40 minutes at 37° C. Afterwards, tissue pieces were dissociated by pipetting into 5 mL pipette and centrifuged at 300 g for 5 min The pellet was suspended in cell growth media Dulbecco's Modified Eagle Medium: Nutrient Mixture F-12 (“DMEM/F12”) (available from Invitrogen, Carlsbad, Calif.) (1:1) containing amphoterecin, penicillin and streptomycin supplemented with 10% fetal bovine serum. Cell suspensions were transferred into T-tissue culture flask and grown until 80-90% confluence. The cells were placed in a T-75 flask before being used for flow analysis and differentiation.

Another embodiment is the use of MSCs from the umbilical cord during harvested during delivery. Once received, the tissue is washed two to three times in sterile PBS and then divided into pieces of approximately 5 grams each. Thereafter, the tissue is decontaminated, and each 5 gram aliquot of tissue is placed in a sterile 100 mm tissue culture dish, and covered with a lid to prevent drying. The tissue was dissociated via enzymatic digestion in 50 cc tubes, and is minced into fragments less than 1 mm³ using a sterile scalpel. Then, the chopped tissue is placed in an enzyme bath, and the tube is capped and transferred to an incubator. The tubes were swirled for fifteen seconds every ten minutes for forty minutes. Thereafter, the digesting enzyme was diluted by adding 45 mL of cold DME/F12 complete media (FBS, Pen/Strep and Amphotericin B), with the tubes being capped and inverted to mix the contents. Next, the tubes were centrifuged at 400×g for fifteen minutes on low break. The top media is aspirated using a 25 mL pipette by leaving approximately 5 mL at the bottom of the tube, with special care being taken to aspirate the entire medium in the tube. The bottom 5 mL medium (containing tissue fragments and cells including MSCs) was resuspended in fresh 20 mL DME-F12 complete medium mixed well and placed into a t-75 flask, and transferred to an incubator. The tissue is washed off during the first media change after 48 hours post-digestion, and the media was changed three times per week. Cells are grown to 70%-80% confluence and then either passaged, frozen down as passage zero cells, or differentiated. Cells were not allowed to reach confluence or to remain at confluence for extended periods of time.

Cell expansion for cells originating from any of the abovementioned tissues above takes place in clean room facilities purpose built for cell therapy manufacture and meeting GMP clean room classification. In a sterile class II biologic safety cabinet located in a class 10,000 clean production suite, cells were thawed under controlled conditions and washed in a 15 mL conical tube with 10 ML of complete DMEM-low glucose media (cDMEM) (GibcoBRL, Grand Island, N.Y.) supplemented with 20% Fetal Bovine Serum (Atlas) from dairy cattle confirmed to have no BSE % Fetal Bovine Serum specified to have Endotoxin level less than or equal to 100 EU/mL (with levels routinely less than or equal to 10 EU/mL) and hemoglobin level less than or equal to 30 mg/dl (levels routinely less than or equal to 25 mg/dl). The serum lot used is sequestered and one lot was used for all experiments.

Cells are subsequently placed in a T-225 flask containing 45 mL of cDMEM and cultured for 24 hours at 37° C. at 5% CO2 in a fully humidified atmosphere. This allowed the MSC to adhere. Non-adherent cells were washed off using cDMEM by gentle rinsing of the flask. This resulted in approximately 6 million cells per initiating T-225 flask. The cells of the first flask were then split into 4 flasks. Cells were grown for 4 days after which approximately 6 million cells per flask were present (24 million cells total). This scheme was repeated but cells were not expanded beyond 10 passages, and were then banked in 6 million cell aliquots in sealed vials for delivery. All processes in the generation, expansion, and product production were performed under conditions and testing that was compliant with current Good Manufacturing Processes and appropriate controls, as well as Guidances issued by the FDA in 1998 Guidance for Industry: Guidance for Human Somatic Cell Therapy and Gene Therapy; the 2008 Guidance for FDA Reviewers and Sponsors Content and Review of Chemistry, Manufacturing, and Control (CMC) Information for Human Somatic Cell Therapy Investigational New Drug Applications (INDs); and the 1993 FDA points-to-consider document for master cell banks were all followed for the generation of the cell products described. Donor cells are collected in sterile conditions, shipped to a contract manufacturing facility, assessed for lack of contamination and expanded. The expanded cells are stored in cryovials of approximately 6 million cells/vial, with approximately 100 vials per donor. At each step of the expansion quality control procedures were in place to ensure lack of contamination or abnormal cell growth.

Without departing from the spirit of the teachings of this application, mesenchymal stem cells may be optimized to possess heightened immune modulatory properties. In one embodiment this may be performed by exposure of mesenchymal stem cells to hypoxic conditions, specifically hypoxic conditions can comprise an oxygen level of lower than 10%. In some embodiments, hypoxic conditions comprise up to about 7% oxygen. For example, hypoxic conditions can comprise up to about 7%, up to about 6%, up to about 5%, up to about 4%, up to about 3%, up to about 2%, or up to about 1% oxygen. In some embodiments, hypoxic conditions comprise about 1% oxygen up to about 7% oxygen. For example, hypoxic conditions can comprise about 1% oxygen up to about 7% oxygen; about 2% oxygen up to about 7% oxygen; about 3% oxygen up to about 7% oxygen; about 4% oxygen up to about 7% oxygen; about 5% oxygen up to about 7% oxygen; or about 6% oxygen up to about 7% oxygen. As another example, hypoxic conditions can comprise about 1% oxygen up to about 7% oxygen; about 1% oxygen up to about 6% oxygen; about 1% oxygen up to about 5% oxygen; about 1% oxygen up to about 4% oxygen; about 1% oxygen up to about 3% oxygen; or about 1% oxygen up to about 2% oxygen. As another example, hypoxic conditions can comprise about 1% oxygen up to about 7% oxygen; about 2% oxygen up to about 6% oxygen; or about 3% oxygen up to about 5% oxygen. As another example, hypoxic conditions can comprise 1% oxygen up to 7% oxygen; 2% oxygen up to 6% oxygen; or 3% oxygen up to 5% oxygen. In some embodiments, hypoxic conditions can comprise no more than about 2% oxygen. For example, hypoxic conditions can comprise no more than 2% oxygen.

### EXAMPLE

6-8 week old C57BL/6 mice are treated with Conconavalin A that was dissolved in pyrogen-free PBS at a concentration of 1 mg/ml and injected intravenously through the tail vein (15 mg/kg). Human umbilical cord blood mononuclear cells isolated by ficoll method are pretreated with hepatocyte growth factor at a concentration of 100 gg/ml for 24 hours followed by washing in PBS. Cells are administered 12 hours after conconavalin A challenge. A group of 10 mice receive cord blood mononuclear cells that are not pretreated, another group receive HGF pretreated cells, and another group receive only conconavalin A challenge. 48 hours after conconavalin A challenge mice are sacrificed. A significant reduction in AST and ALT are seen in the cord blood mononuclear cells as compared to control, regardless of HGF pretreatment. TUNEL staining reveals substantially less apoptotic hepatocytes in the group receiving HGF pretreated cells as compared to cells alone. Animals receiving no cells exhibit the highest amount of apoptotic hepatocytes.

1. A method of treating liver failure through administration of an immunologically active cell population.

2. The method of claim 1, wherein said cell population comprises a mesenchymal stem cell population that is or has been rendered immunologically active.

3. The method of claim 1, wherein said immunologically active cell population is cord blood mononuclear cells.

4. The method of claim 3, wherein said cord blood mononuclear cells are treated with an immune modulator prior to administration.

5. The method of claim 4, wherein said cord blood mononuclear cells are cultured with an immune modulator prior to administration.

6. The method of claim 5, wherein said culture with said immune modulator is of time course sufficient to induce ability to inhibit proliferation of an activated T cell.

7. The method of claim 5, wherein said culture with said immune modulator is of time course sufficient to induce ability to inhibit interferon gamma production of an activated T cell.

8. The method of claim 4, wherein said immune modulator is selected from a group comprising of: IL-4, IL-10, IL-13, IL-20, TGF-beta, CXCL12, and inhibin.

9. The method of claim 8, wherein said immune modulator is TGF-beta.

10. The method of claim 4, wherein said immune modulator is a combination of TGF-beta, VEGF, and PGE-2.

11. The method of claim 1, wherein said immunologically active cell population is selected from a group of cells comprising of: a) mesenchymal stem cells; b) T regulatory cells; c) type 2 monocytes; d) CD5 positive B cells; e) type 2 NKT cells; f) tolerogenic dendritic cells; g) gamma delta T cells; h) T cells with immune regulatory properties; i) CD34 cells; j) very small embryonic like stem cells and k) Sertoli cells.

12. The method of claim 11, wherein said mesenchymal stem cell is derived from tissue comprising a group selected from: a) Wharton's Jelly; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; o) periventricular fluid; and p) placental tissue.

13. The method of claim 12, wherein said mesenchymal stem cells express a marker or plurality of markers selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

14. The method of claim 13, wherein said mesenchymal stem cells do not express substantial levels of HLA-DR, CD117, and CD45.

15. The method of claim 11, wherein said mesenchymal stem cells are generated from a pluripotent stem cell.

16. The method of claim 15, wherein said pluripotent stem cell is selected from a group comprising of: a) an embryonic stem cell; b) an inducible pluripotent stem cell; c) a parthenogenic stem cell; and d) a somatic cell nuclear transfer derived stem cell.

17. The method of claim 16, wherein said embryonic stem cell population expresses genes selected from a group comprising of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

18. The method of claim 16, wherein said inducible pluripotent stem cell possesses markers selected from a group comprising of: CD10, CD13, CD44, CD73, CD90, PDGFr-alpha, PD-L2, and HLA-A,B,C and possesses ability to undergo at least 40 doublings in culture, while maintaining a normal karyotype upon passaging.

19. The method of claim 16, wherein said parthenogenic stem cells wherein said parthenogenically derived stem cells are generated by addition of a calcium flux inducing agent to activate an oocyte followed by enrichment of cells expressing markers selected from a group comprising of SSEA-4, TRA 1-60 and TRA 1-81.

20. The method of claim 16, wherein said somatic cell nuclear transfer derived stem cells possess a phenotype negative for SSEA-1 and positive for SSEA-3, SSEA-4, TRA-1-60, TRA-1-81, and alkaline phosphatase.
